(19) United States
(12) Patent Application Publication
(10) Pub. No.: US 2026/0306378 A1
(43) Pub. Date: Oct. 8, 2026

(54) FOAMING CLEANSING COMPOSITION WITH LONG CHAIN SURFACTANT SYSTEM

(71) Applicant: L'OREAL, Paris (FR)

(72) Inventors: Christopher PANG, New York, NY (US); Mariko Hasebe, New York, NY (US); Changlong Chen, Edison, NJ (US); Hungta Lin, Teaneck, NJ (US); Lingwei Xu, Shanghai (CN)

(73) Assignee: L'OREAL, Paris (FR)

(21) Appl. No.: 19/093,782

(22) Filed: Mar. 28, 2025

(51) Int. Cl. A61K 8/46; A61K 8/44; A61K 8/60; A61K 8/68; A61K 8/88; A61Q 1/14; A61Q 5/02; A61Q 19/10

(52) U.S. Cl. CPC A61K 8/466; A61K 8/442; A61K 8/604; A61K 8/68; A61K 8/88; A61Q 1/14; A61Q 5/02; A61Q 19/10; A61K 2800/30; A61K 2800/596; A61K 2800/70

(57) ABSTRACT

A foaming cleansing composition, comprising a blend of surfactants comprising a combination of at least two amphoteric surfactants, including cocoamidopropyl hydroxysultaine and coco betaine, present in a total amount of about 8% by weight, based on the weight of the composition and a combination of at least two anionic surfactants, comprising sodium methyl cocoyl taurate and at least one of sodium cocoyl sarcosinate or sodium lauroyl sarcosinate, each present in an amount of at least 1.8% by weight or 1.9% by weight, based on the weight of the composition, respectively, and water wherein the composition demonstrates long-term stability at elevated temperatures, remaining visibly clear with stable viscosity, antimicrobial resistance, and resistance to cloudiness and sedimentation at ambient and high temperatures.

### FIELD

This invention relates to a foaming cleansing composition that includes a long chain surfactant system for viscosity, clarity and antimicrobial benefit.

### BACKGROUND

Foaming cleansing compositions are widely used in personal care products due to their ability to effectively cleanse while generating an appealing foam. Traditionally, such compositions include various surfactants to achieve desired foaming and cleansing properties.

Many conventional cleansing systems rely on polyethylene glycol based ingredients, including PEG-type thickeners, as well as sulfate surfactants, and amphoteric surfactants to build viscosity. There are challenges in reducing the amounts of or replacing ingredients such as PEG-type thickeners and sulfate surfactants due to issues with loss of viscosity. Compositions that lack PEG-type thickeners and sulfate surfactants can suffer from drawbacks such as instability at higher temperatures, leading to cloudiness or sedimentation, and inconsistent antimicrobial properties. Moreover, the viscosity of these compositions can vary significantly with temperature changes, affecting user experience and product performance. Therefore, there is a need for an improved foaming cleansing composition that overcomes these limitations while reducing or eliminating certain know or perceived harsh and undesirable thickening ingredients.

The present invention aims to address these challenges by providing a foaming cleansing composition that remains visibly clear with stable viscosity, antimicrobial resistance, and resistance to cloudiness and sedimentation at both ambient and high temperatures with desirable foaming and cleansing properties without reliance on added thickeners surfactants and, particularly PEG-type polymeric thickeners and sulfate surfactants.

### SUMMARY

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

The disclosure provides, in various embodiments, a foaming cleansing composition comprising a long chain surfactant system for viscosity, clarity and antimicrobial benefit that includes at least two amphoteric surfactants, including cocoamidopropyl hydroxysultaine and coco betaine, present in a total amount of about 8% by weight; a combination of at least two anionic surfactants, comprising sodium methyl cocoyl taurate and at least one of sodium cocoyl sarcosinate or sodium lauroyl sarcosinate, each present in an amount of at least 1.8% by weight or 1.9% by weight, respectively, all amounts by weight, based on the weight of the composition; wherein the composition demonstrates long-term stability at elevated temperatures, remaining visibly clear with stable viscosity, antimicrobial resistance, and resistance to cloudiness and sedimentation at ambient and high temperatures. The foaming cleansing composition includes water. The foaming cleansing composition may be essentially free of added PEG-type polymeric thickeners. In some embodiments, the foaming cleansing composition demonstrates antimicrobial activity against C. acnes, S. aureus, and S. epidermidis. In some embodiments, the foaming cleansing composition comprises the ratio of sodium cocoyl sarcosinate to sodium methyl cocoyl taurate in a range from about 1:1 to about >1:1 by weight of each ingredient.

According to an embodiment of the invention, a foaming cleansing composition comprises a blend of surfactants comprising a combination of at least two amphoteric surfactants, including cocoamidopropyl hydroxysultaine and coco betaine, present in a total amount of about 8% by weight; a combination of at least two anionic surfactants, comprising sodium methyl cocoyl taurate and at least one of sodium cocoyl sarcosinate or sodium lauroyl sarcosinate, each present in an amount of at least 1.8% by weight or 1.9% by weight, respectively; wherein the composition demonstrates long-term stability at elevated temperatures, remaining visibly clear with stable viscosity, antimicrobial resistance, and resistance to cloudiness and sedimentation at ambient and high temperatures.

In some embodiments, the foaming cleansing composition comprises cocoamidopropyl hydroxysultaine present in a range from about 1% to about 20% and coco betaine present in a range from about 1% to about 15%. In some embodiments, the foaming cleansing composition comprises cocoamidopropyl hydroxysultaine present in a range from about 3% to about 7%, and coco betaine present in a range from about 1% to about 5%.

In some embodiments, the foaming cleansing composition comprises water present in a range from about 40% to about 90%, by weight, based on the weight of the foaming cleansing composition.

In some embodiments, the foaming cleansing composition comprises sodium methyl cocoyl taurate present from about 0.1% to about 1.5%, or from about 0.5% to about 1.2%, or about 0.9%. In some embodiments, the foaming cleansing composition comprises sodium cocoyl sarcosinate present in an amount of at least about 1.8%, or from about 0% to about 6%, or from about 1.8% to about 10%, or from about 4.5% to about 10%, or about 4.5%. In some embodiments, the foaming cleansing composition comprises the ratio of sodium cocoyl sarcosinate to sodium methyl cocoyl taurate in a range from about 1:1 to about >1:1 by weight of each ingredient.

In some embodiments, the foaming cleansing composition comprises sodium lauroyl sarcosinate present from about 0% to about 6%, or from about 1.9% to about 6%, or about 4.5%.

In some particular embodiments, the foaming cleansing composition comprises comprising or sodium lauroyl sarcosinate wherein the ratio of sodium lauroyl sarcosinate to sodium methyl cocoyl taurate is in a range from about 1:1 to about >1:1 by weight of each ingredient.

In some embodiments, the foaming cleansing composition comprises at least one additional anionic surfactant comprising sodium isethionate present from about 0.001% to about 2%.

In some embodiments, the foaming cleansing composition comprises at least one additional anionic surfactant comprising sodium lauroyl lactylate present from about 0.005% to about 0.01%, or from about 0.015% to about 0.035%, or from about 0.02% to about 0.03%, or about 0.025%.

In some embodiments, the foaming cleansing composition demonstrates each of foaming, viscosity, and solubility stability at ambient and elevated temperatures, and antimicrobial activity against C. acnes, S. aureus, and S. epidermidis.

In some embodiments, the foaming cleansing composition comprises at least one or a combination of polylysine, ceramide, and glycolipids. In some embodiments, the foaming cleansing composition comprises a glycolipid selected from the group consisting of rhamnolipids, trehalolipids, sophorolipids, mannosylerythritol lipids, and combinations thereof. In some embodiments, the foaming cleansing composition comprises a glycolipid comprising at least one rhamnolipid.

In some embodiments, the foaming cleansing composition comprises one or more ingredients selected from the group consisting of ceramide EOP, ceramide NP, ceramide AP, caprylyl glycol, phenoxyethanol, PEG-150 pentaerythrityl tetrastearate, carbomer, xanthan gum, propanediol, tetrasodium glutamate diacetate, sodium benzoate, phytosphingosine, glycerin, cholesterol, benzoic acid, sodium chloride, citric acid, triethyl citrate, sodium hyaluronate, niacinamide.

In some embodiments, the foaming cleansing composition comprises PEG-150 pentaerythrityl tetrastearate in an amount that is less than 1%. In some embodiments, the foaming cleansing composition excludes PEG/PEO polymeric thickeners, parabens, and combinations thereof. In some embodiments, the foaming cleansing composition excludes PEG-150 pentaerythrityl tetrastearate, PEG-6 caprylic/capric glycerides, or a combination thereof.

According to an embodiment of the invention, a foaming cleansing composition, comprises a blend of surfactants comprising cocoamidopropyl hydroxysultaine in a range from about 3% to about 7%; a combination of at least two anionic surfactants, including sodium methyl cocoyl taurate present from about 0.1% to about 1.5%, or from about 0.5% to about 1.2%, or about 0.9%, and sodium cocoyl sarcosinate present in an amount of at least about 1.8%, or from about 0% to about 6%, or from about 1.8% to about 10%, or from about 4.5% to about 10%, or about 4.5%; wherein the composition demonstrates long-term stability at elevated temperatures, remaining visibly clear with stable viscosity, antimicrobial resistance, and resistance to cloudiness and sedimentation at ambient and high temperatures. The foaming cleansing composition includes water.

According to an embodiment of the invention, a foaming cleansing composition, comprises a blend of surfactants comprising a combination of at least two amphoteric surfactants, including cocoamidopropyl hydroxysultaine and coco betaine, present in a total amount of about 8% by weight; a combination of at least two anionic surfactants, comprising sodium methyl cocoyl taurate and at least one of sodium cocoyl sarcosinate or sodium lauroyl sarcosinate, each present in an amount of at least 1.8% by weight or 1.9% by weight, respectively; wherein water is present in a range from about 40% to about 90%, by weight, based on the weight of the foaming cleansing composition; wherein the composition demonstrates foaming, viscosity, and solubility stability at ambient and elevated temperatures, and antimicrobial activity against C. acnes, S. aureus, and S. epidermidis; comprising one or more ingredients selected from the group consisting of ceramide EOP, ceramide NP, ceramide AP, caprylyl glycol, phenoxyethanol, PEG-150 pentaerythrityl tetrastearate, carbomer, xanthan gum, propanediol, tetrasodium glutamate diacetate, sodium benzoate, phytosphingosine, glycerin, cholesterol, benzoic acid, sodium chloride, citric acid, triethyl citrate, sodium hyaluronate, niacinamide; which comprises PEG-150 pentaerythrityl tetrastearate in an amount that is less than 1%. The foaming cleansing composition includes water.

The foaming cleansing composition offers several technical advantages: it maintains stability and clarity under varying temperature conditions; allows flexibility in formulation while ensuring effective surfactant performance; optimizes surfactant concentrations for improved foaming and cleansing; ensures hydration and consistency; offers natural surfactant options for gentle cleansing and effective microbial control; minimizes thickeners while maintaining texture; and provides formulations free from certain additives for sensitive skin; and reduces potential irritants.

These and other aspects of the invention are set out in the appended claims, and described in greater detail in the detailed description of the invention.

It is to be understood that both the foregoing general description and the following detailed description are exemplary and explanatory only and are not restrictive of the disclosure.

### DETAILED DESCRIPTION

The disclosure provides, in various embodiments, a foaming cleansing composition comprising a long chain surfactant system for viscosity, clarity and antimicrobial benefit that includes at least two amphoteric surfactants, including cocoamidopropyl hydroxysultaine and coco betaine, present in a total amount of about 8% by weight; a combination of at least two anionic surfactants, comprising sodium methyl cocoyl taurate and at least one of sodium cocoyl sarcosinate or sodium lauroyl sarcosinate, each present in an amount of at least 1.8% by weight or 1.9% by weight, respectively; wherein the composition demonstrates long-term stability at elevated temperatures, remaining visibly clear with stable viscosity, antimicrobial resistance, and resistance to cloudiness and sedimentation at ambient and high temperatures. The foaming cleansing composition may be essentially free of added PEG-type polymeric thickeners. In some embodiments, the foaming cleansing composition demonstrates antimicrobial activity against C. acnes, S. aureus, and S. epidermidis. In some embodiments, the foaming cleansing composition comprises the ratio of sodium cocoyl sarcosinate to sodium methyl cocoyl taurate in a range from about 1:1 to about >1:1 by weight of each ingredient. The foaming cleansing composition includes water.

The key problem in the art to be solved is to develop a surfactant combination to build viscosity without the need to rely on PEG as the main thickener in the formulation, and thus avoid the negative skin effects of PEG based ingredients. Referencing the examples herein, the inventors surprisingly found that use of sodium cocoyl sarcosinate, which is more typically used in sun and skin care compositions in amounts well below 5%, in amounts of at least 8.5% and higher demonstrated excellent performance in maintaining long term stability at elevated temperature and provided improved solubility with minimal sedimentation and minimal to no loss of clarity. The inventors found that combining sodium cocoyl sarcosinate with or replacing sodium cocoyl sarcosinate with sodium lauroyl sarcosinate provides good results. Exemplified inventive compositions demonstrate stability in viscosity and visual clarity, as well as excellent antimicrobial benefits in a pleasing foaming cleanser format. The inventors have demonstrated that the inventive composition provides cleansing capabilities on par with current benchmark.

The foaming cleansing composition is shelf stable and stable on application, demonstrating a viscosity of about 18-25 UD (Viscosity Rheomat M3 Spindle), and a pH about 4.5 to about 6.5, or about pH 5. The foaming cleansing composition may be essentially free or devoid of added PEG-type, PEG/PEO polymeric thickeners.

The foaming cleansing composition offers several technical advantages: it maintains stability and clarity under varying temperature conditions; allows flexibility in formulation while ensuring effective surfactant performance; optimizes surfactant concentrations for improved foaming and cleansing; ensures hydration and consistency; provides specific concentration ranges for targeted cleansing efficacy; allows variation in surfactant concentration to suit different formulation needs; maintains surfactant balance for optimal performance; offers alternative surfactant options for enhanced cleansing; enhances formulation mildness; improves emulsification properties; provides robust performance across various conditions; enhances moisturizing and protective properties; offers natural surfactant options for gentle cleansing and effective microbial control; enhances versatility and efficacy; minimizes thickeners while maintaining texture; provides formulations free from certain additives for sensitive skin; and reduces potential irritants.

In various embodiments, the foaming cleansing composition may be a skin cleanser, shampoo, or body wash and in some particular embodiments, the foaming cleansing composition is a facial cleanser and/or makeup remover.

The foaming cleansing composition, can comprise, consist of, or consist essentially of the essential elements and limitations of the invention described herein, as well as any additional or optional ingredients, components, or limitations described herein or otherwise useful.

“Cleanser” or “foaming cleansing composition” as used herein means and refers to any foaming cleansing composition utilized for application to a keratinous tissue for one or more of cleansing the skin, removal of make-up and the like.

“Cosmetically acceptable” as used herein means and refers to a carrier that is compatible with any keratinous substrate.

As used herein with respect to the foaming property of cleansing formulations, including the foaming cleansing composition, the term “foaming” means and refers to the capacity of a composition (inventive or comparative) to develop a foam upon rubbing that would be associated with applying and gently scrubbing skin or other keratinous substrates with the formulation. Reference is made to a known testing method for establishing foam formation in vitro employing equipment that is well known in the art, namely analysis using a Krauss DFA 100™ Dynamic Foam Analyzer, which provides a relative measure of achieved foam formation, as more specifically described herein.

“Keratinous substrate” and “keratinous tissue” each includes but is not limited to skin, hair, and nails.

“Skin” as used herein means and refers to skin materials containing keratin such as facial and body skin, scalp, eyebrows, and lips.

“Stable” and “Stability” as used herein means that the foaming cleansing composition does not show any signs of significant changes in one or more of color, odor, pH, visible clarity (minimal or no cloudiness or sedimentation or precipitation) or viscosity at room temperature and at temperatures in the range from about 25° C. to about 45° C. In various embodiments, an inventive composition remains stable at temperatures in the range from about 25° C. to about 45° C., over a time period of at least one month, or at least two months, or longer, or any value, range, or sub-range therebetween. In some embodiments, a foaming cleansing composition is stable at 25° C. for at least one month, or at least two months, or longer, or any value, range, or sub-range there between. In some embodiments, a foaming cleansing composition is stable at 45° C. for at least one month, or at least two months, or longer, or any value, range, or sub-range therebetween. Viscosity was measured in the UD units by viscometer Rheomat R180 using an M3 spindle for −10 minutes at 25° C. Stable inventive compositions demonstrate viscosity in a range from about 15 UD to about 50 UD (M3) based on use of RM180 Rheomat M3 Spindle analysis. In some particular embodiments, the viscosity is in the range from about 20 UD to about 40 UD. Unsatisfactory compositions are deemed unstable at viscosities that fall below 12 UD after exposure to 45° C. for after two months.

### Foaming Cleansing Composition

The disclosure provides, in various embodiments, a foaming cleansing composition comprising a long chain surfactant system for viscosity, clarity and antimicrobial benefit that includes at least two amphoteric surfactants, including cocoamidopropyl hydroxysultaine and coco betaine, present in a total amount of about 8% by weight; a combination of at least two anionic surfactants, comprising sodium methyl cocoyl taurate and at least one of sodium cocoyl sarcosinate or sodium lauroyl sarcosinate, each present in an amount of at least 1.8% by weight or 1.9% by weight, and may optionally include amphoteric surfactants in addition to cocoamidopropyl hydroxysultaine and coco betaine, and may optionally include anionic surfactants in addition to sodium methyl cocoyl taurate and at least one of sodium cocoyl sarcosinate or sodium lauroyl sarcosinate. In some embodiments, the foaming cleansing composition may comprise sodium lauroyl sarcosinate, sodium isethionate, or a combination thereof. In some embodiments, the foaming cleansing composition may comprise at least one glycolipid selected from rhamnolipids, ceramides, polylysine, or a combination thereof. Rhamnolipids are glycolipids produced by various bacterial species. They consist of one rhamnose fragment (mono-rhamnolipid) or of two rhamnose fragments (di-rhamnolipid) linked by a glycosidic bond to one, two or three chains of β-hydroxylated fatty acids linked to one another by an ester bond. Use may be made, as rhamnolipid, of the one sold under the name Rheance One by Evonik (INCI name: glycolipids).

In some embodiments, the foaming cleansing composition comprises sodium lauroyl sarcosinate present from about 0% to about 6%, or from about 1.9% to about 6%, or about 4.5%. In some embodiments, the foaming cleansing composition comprises sodium isethionate present from about 0.001% to about 2%.

In some embodiments, the foaming cleansing composition comprises one or more of rhamnolipids present from about 0% to about 1%, or from about 0.5% to about 0.5%, or polylysine present from about 0% to about 1%, or from about 0.005% to about 0.0.1%, or a combination thereof, the amounts representing an exemplary embodiment, by weight, based on the weight of the composition.

In some embodiments, the foaming cleansing composition may include or excludes any one or more of nonionic or cationic surfactants, and in particular embodiments, may excluded sulfate, PEG/POE, or a combination of such surfactants.

In general, as described and exemplified herein, all amounts given are by weight, based on the weight of the composition, unless otherwise stated, such as for ratios between two ingredients, which are typically based on the weights of each ingredient.

### Amphoteric Surfactants

In various embodiments, the foaming cleansing composition comprises at least two amphoteric surfactants (or zwitterionic surfactant).

In some embodiments, the least two amphoteric surfactants comprise cocoamidopropyl hydroxysultaine and coco betaine, which are present in a total amount of about 8% by weight.

In various embodiments, the amount of cocamidopropyl cocoamidopropyl hydroxysultaine is from about 1% to about 20%, or from about 3% to about 7%, and coco betaine is from about 1% to about 15%, or from about 1% to about 5%, or any suitable combination, sub-combination, range, or sub-range thereof by weight, based on the weight of the foaming cleansing composition. One of ordinary skill in the art, however, will appreciate that other ranges are within the scope of the invention. Thus, each of the amphoteric surfactants comprising at least cocoamidopropyl hydroxysultaine and coco betaine, are present by weight, based on the total weight of the foaming cleansing composition, from about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, to about 20 weight percent, including increments and ranges therein and therebetween.

In some embodiments, the foaming cleansing composition comprises one or more additional amphoteric surfactants.

In some embodiments, one or more additional amphoteric surfactant, if present, may be selected from, for example, betaines, alkyl sultaines, alkyl amphoacetates and alkyl amphodiacetates, alkyl amphoproprionates, amphocarboxylates, alkyl betaines, amidoalkyl betaines, amphophosphates, phosphobetaines, pyrophosphobetaines, carboxyalkyl polyamines, amidoalkyl sultaines, salts thereof, or mixtures thereof.

Betaines which can be used in the current compositions include those having the formulas below:

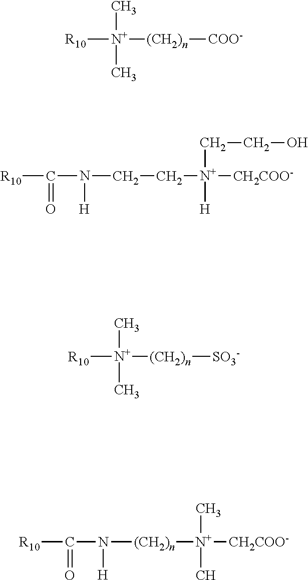

wherein R¹⁰ is an alkyl group having 8-18 carbon atoms; and n is an integer from 1 to 3.

Particularly useful betaines include, for example, coco betaine, cocoamidopropyl betaine, lauryl betaine, laurylhydroxy sulfobetaine, lauryldimethyl betaine, cocoamidopropyl hydroxysultaine, behenyl betaine, capryl/capramidopropyl betaine, lauryl hydroxysultaine, stearyl betaine, and mixtures thereof. Typically, the at least one betaine compound is selected from the group consisting of coco betaine, cocoamidopropyl betaine, behenyl betaine, capryl/capramidopropyl betaine, lauryl betaine, and mixtures thereof, and more typically coco betaine.

Hydroxyl sultaines useful in the compositions of the invention include the following

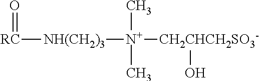

wherein R is an alkyl group having 8-18 carbon atoms.

More specific examples include, but are not limited to cocamidopropyl hydroxysultaine, lauryl hydroxysultaine, or mixtures thereof.

Useful alkylamphoacetates include those having the formula

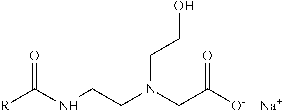

wherein R is an alkyl group having 8-18 carbon atoms.

Useful alkyl amphodiacetates include those having the formula

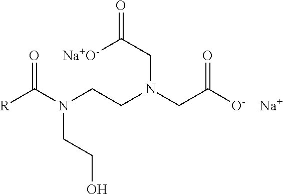

wherein R is an alkyl group having 8-18 carbon atoms.

### Alkyl Amphopropionates

Exemplary and non-limiting examples of useful alkyl amphopropionates include cocoamphopropionate, caprylamphopropionate, cornamphopropionate, caproampho-propionate, oleoamphopropionate, isostearoamphopropionate, stearoamphopropionate, lauroamphopropionate, salts thereof, or mixtures thereof.

The one or more additional amphoteric surfactant of the present disclosure may be optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

The one or more additional amphoteric surfactant may be present in the foaming cleansing composition in a range from about 0.1% to about 20%, based on the weight of the foaming cleansing composition.

In various embodiments, each of one or more additional amphoteric surfactant may be present in an amount in the range of from about 0.1% to about 20%, and the total amount of surfactant may be present in a range from about 0.1% to about 20%. In some embodiments, each of one or more additional amphoteric surfactant is present in an amount that is from about 1% to about 20%, or from about 1% to about 15%, or from about 3% to about 10%, or from about 5% to about 7%, or any value, range, or sub-range therebetween by weight, based on the weight of the foaming cleansing composition.

In some embodiments, one or more additional amphoteric surfactant includes cocamidopropyl cocoamidopropyl hydroxysultaine present in the foaming cleansing composition in a range from about 1% to about 10%.

Thus, in various embodiments, each of one or more additional amphoteric surfactant may be present in a composition according to the disclosure from 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 to about 20 percent, including increments and ranges therein and there between.

### Anionic Surfactants

In various embodiments, the foaming cleansing composition comprises at least two anionic surfactants comprising sodium methyl cocoyl taurate and at least one of sodium cocoyl sarcosinate present in an amount of at least 1.8%, or sodium lauroyl sarcosinate present in an amount of at least 1.9%, by weight, respectively.

In some particular embodiments, the foaming cleansing composition comprises at least two anionic surfactants comprising sodium methyl cocoyl taurate and sodium cocoyl sarcosinate present in an amount of at least 1.8% by weight.

In some particular embodiments, the foaming cleansing composition comprises at least two anionic surfactants comprising sodium methyl cocoyl taurate and sodium lauroyl sarcosinate present in an amount of at least 1.9% by weight.

In some particular embodiments, the foaming cleansing composition comprises sodium cocoyl sarcosinate wherein the ratio of sodium cocoyl sarcosinate to sodium methyl cocoyl taurate is in a range from about 1:1 to about >1:1 by weight of each ingredient.

In some particular embodiments, the foaming cleansing composition comprises sodium lauroyl sarcosinate wherein the ratio of sodium lauroyl sarcosinate to sodium methyl cocoyl taurate is in a range from about 1:1 to about >1:1 by weight of each ingredient.

In some particular embodiments, the foaming cleansing composition comprises sodium lauroyl sarcosinate present from about 1.9% to about 6%, by weight, based on the weight of the composition.

In some embodiments, the foaming cleansing composition comprises one or more anionic surfactants in addition to sodium methyl cocoyl taurate and one or both of sodium cocoyl sarcosinate and sodium lauroyl sarcosinate.

In some embodiments, the composition includes one or a combination of sodium isethionate and sodium lauroyl lactylate. In some embodiments, the composition includes at least one of sodium isethionate present from about 0.001% to about 2%, sodium lauroyl lactylate present from about 0.005% to about 0.01%, or from about 0.015% to about 0.035%, or from about 0.02% to about 0.03%, or about 0.025%, or a combination thereof.

In some particular embodiments, the foaming cleansing composition comprises at least one additional anionic surfactant comprising sodium isethionate is present from about 0.001% to about 2%, by weight, based on the weight of the composition.

In some particular embodiments, the foaming cleansing composition comprises at least one additional anionic surfactant comprising sodium lauroyl lactylate is present from about 0.015% to about 0.035%, by weight, based on the weight of the composition.

These surfactants may be present in specific amounts, with sodium methyl cocoyl taurate from about 0.1% to about 1.5%, or from about 0.5% to about 1.2%, or about 0.9%, sodium cocoyl sarcosinate from at least about 1.8%, or from about 0% to about 6%, and sodium lauroyl sarcosinate from about 1.9% to about 6%, or about 4.5%.

In various embodiments, the amount of each anionic surfactant is present from about 0.1% to about 6%, or from about 1.8% to about 4.5%, or any suitable combination, sub-combination, range, or sub-range thereof by weight, based on the weight of the foaming cleansing composition.

One of ordinary skill in the art, however, will appreciate that other ranges are within the scope of the invention.

Thus, each one of the anionic surfactants, may be present by weight, based on the total weight of the foaming cleansing composition, from about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, to about 6.0 weight percent, including increments and ranges therein and therebetween.

In some embodiments, the foaming cleansing composition comprises one or more additional anionic surfactants. More generally, at least one additional anionic surfactant, if present, may be selected from non-sulfate anionic surfactants, sulfate anionic surfactants, their salts thereof, or mixtures thereof.

### Non-Sulfate Anionic Surfactants

When present, the total amount of non-sulfate anionic surfactants in the foaming cleansing compositions can vary but typically ranges from about 4% to about 30%, based on the total weight of the foaming cleansing composition. In some cases, the total amount of non-sulfate anionic surfactants in the foaming cleansing composition may be from about 4% to about 25%, or from about 5% to about 25%, or from about 5% to about 23%, or from about 6% to about 24%, or from about 6% to about 25%, or from about 7% to about 25%, or from about 8% to about 25%, or from about 10% to about 23%, or any suitable combination, sub-combination, range, or sub-range thereof by weight, based on the weight of the foaming cleansing composition based.

In various embodiments, the total amount of non-sulfate anionic surfactants in the foaming cleansing compositions, if present, is typically at about 6, 6.5, 7, 7.5, 8, 8.5, 8.8, 9, 9.5, 10, 10.5, 11, 11.4, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, to about 25, weight percent based on the total weight of the foaming cleansing composition, including increments and ranges therein and there between.

Useful non-sulfate anionic surfactants include, but are not limited to, alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, or mixtures thereof. Non-limiting examples of useful non-sulfate anionic surfactants are provided below.

### Acyl Isethionates

Non-limiting examples of useful acyl isethionates and their salts include those of formula (III) and (IV):

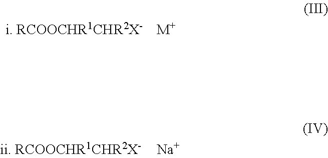

wherein R, R¹, and R² are each independently chosen from H or an alkyl chain having 1-24 carbon atoms, said chain being saturated or unsaturated, linear or branched; X is COO⁻ or SO₃; and M is any suitable cation.

Although the cation may be chosen from any suitable cation as in formula (III), including, for example, alkali metal ion such as sodium or potassium, ammonium ions, or alkanolammonium ions such as monoethanolammonium or triethanolammonium ions, sodium is a cation, as in formula (IV). In various embodiments, RCO— represents the coconut acid moiety. Non-limiting examples of acyl isethionates include sodium cocoyl isethionate, sodium lauroyl isethionate, sodium lauroyl methyl isethionate, and sodium cocoyl methyl isethionate.

### Acyl Sarcosinates

Non-limiting examples of acyl sarcosinates and their salts include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, and ammonium lauroyl sarcosinate.

In some embodiments, the total amount of acyl sarcosinates in the foaming cleansing composition, if present, is from about 0.5 to about 10%, all amounts by weight based on the weight of the foaming cleansing composition. In some instance, the total amount of acyl sarcosinates in the foaming cleansing composition is from about 0.5 to about 10%, about 1 to about 8%, about 1.5 to about 7%, about 1.75 to about 6%, 2 to about 5%, about 2 to about 4%, about 2.5 to about 4%, about 2.5 to about 3.5%, or about 2.7 to about 3%, all amounts by weight based on the weight of the foaming cleansing composition, including all ranges and subranges therebetween.

### Alkyl Sulfonates

Useful alkyl sulfonates and their salts include alkyl aryl sulfonates, primary alkane disulfonates, alkene sulfonates, hydroxyalkane sulfonates, alkyl glyceryl ether sulfonates, alpha-olefinsulfonates, sulfonates of alkylphenolpolyglycol ethers, alkylbenzenesulfonates, phenvlalkanesulfonates, alpha-olefinsulfonates, olefin sulfonates, alkene sulfonates, hydroxyalkanesulfonates and disulfonates, secondary alkanesulfonates, paraffin sulfonates, ester sulfonates, sulfonated fatty acid glycerol esters, and alpha-sulfo fatty acid methyl esters including methyl ester sulfonate.

In some instances, an alkyl sulfonate of formula (V) is particularly useful:

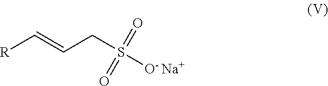

wherein R is selected from H or alkyl chain that has 1-24 carbon atoms, 6-24 carbon atoms, more, 8 to 20 carbon atoms, said chain being saturated or unsaturated, linear or branched, substituted or unsubstituted. Sodium is shown as the cation in the above formula (V) but the cation may be an alkali metal ion such as sodium or potassium, ammonium ions, or alkanolammonium ions such as monoethanolammonium or triethanolammonium ions.

In some instances, the alkyl sulfonate(s) are selected from C₈-C₁₆ alkyl benzene sulfonates, C₁₀-C₂₀ paraffin sulfonates, C₁₀-C₂₄ olefin sulfonates, salts thereof, or mixtures thereof. A non-limiting but particularly useful example of a C₁₀-C₂₄ olefin sulfonate that can be used in the instant compositions is sodium C₁₄₋₁₆ olefin sulfonate.

### Alkyl Sulfosuccinates

Non-limiting examples of useful alkyl sulfosuccinates and their salts include those of formula (VI):

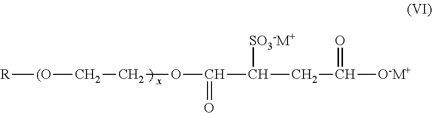

wherein R is a straight or branched chain alkyl or alkenyl group having 10 to 22 carbon atoms, 10 to 20 carbon atoms; x is a number that represents the average degree of ethoxylation, and can range from 0 to about 5, from 0 to about 4, and most from about 2 to about 3.5; and M, which can be the same or different, is chosen from any suitable monovalent cation.

Preferred cations are alkali metal ions such as sodium or potassium, ammonium ions, or alkanolammonium ions such as monoethanolammonium or triethanol-ammonium ions.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, or mixtures thereof.

### Alkyl Sulfoacetates

Non-limiting examples of alkyl sulfoacetates and their salts include, for example, alkyl sulfoacetates such as C4-C18 fatty alcohol sulfoacetates and/or salts thereof, for example, sodium lauryl sulfoacetate. Useful cations for the salts include alkali metal ions such as sodium or potassium, ammonium ions, or alkanolammonium ions such as monoethanolammonium or triethanolammonium ions.

### Alkoxylated Monoacids

Non-limiting examples of alkoxylated monoacids include compounds corresponding to formula (VII):

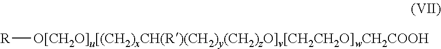

wherein R is a hydrocarbon radical containing from about 6 to about 40 carbon atoms; R′ represents hydrogen or alkyl; u, v, and w, which may be identical or different, independently represent numbers from 0 to 60; x, y, and z, which may be identical or different, independently represent numbers from 0 to 13; and the sum of x+y+z>0.

Compounds corresponding to formula (VII) can be obtained by alkoxylation of alcohols R—OH with ethylene oxide as the sole alkoxide or with several alkoxides and subsequent oxidation. The numbers u, v, and w each represent the degree of alkoxylation. Whereas, on a molecular level, the numbers u, v, and w and the total degree of alkoxylation can only be integers, including zero, on a macroscopic level they are mean values in the form of broken numbers.

In formula (VII), R is linear or branched, acyclic or cyclic, saturated or unsaturated, aliphatic or aromatic, substituted or unsubstituted. Typically, R is a linear or branched, acyclic C6-C40 alkyl or alkenyl group or a C1-C40 alkyl phenyl group, more typically a C8-C22 alkyl or alkenyl group, or a C4-C18 alkyl phenyl group, and even more typically a C12-C18 alkyl group or alkenyl group or a C6-C16 alkyl phenyl group. Further, u, v, w, independently of one another, are typically chosen from a number ranging from 2 to 20, more typically a number ranging from 3 to 17, and most typically a number ranging from 5 to 15. Further still, x, y, z, independently of one another, are typically chosen from a number ranging from 0 to 13, more typically a number ranging from 1 to 10, and most typically a number ranging from 2 to 8.

Suitable alkoxylated monoacids include, but are not limited to: Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C9-11 Pareth-6 Carboxylic Acid, C11-15 Pareth-7 Carboxylic Acid, C12-13 Pareth-5 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C14-15 Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid, or mixtures thereof.

### Acyl Amino Acids

Acyl amino acids that may be used include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. The most common cation associated with the acyl amino acid can be sodium or potassium. Alternatively, the cation can be an organic salt such as triethanolamine (TEA) or a metal salt.

Non-limiting examples of useful acyl amino acids include those of formula (VIII):

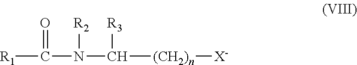

wherein R¹, R², and R³ are each independently selected from H or an alkyl chain having 1-24 carbon atoms, said chain being saturated or unsaturated, linear or branched, substituted or unsubstituted; n ranges from 0 to 30; and X is COO⁻ or SO₃⁻.

In various embodiments, RCO— represents the coconut acid moiety. A non-limiting example is sodium cocoyl glycinate.

### Acyl Taurates

Non-limiting examples of acyl taurates include those of formula (IX):

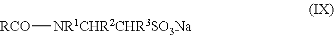

wherein R, R¹, R², and R³ are each independently selected from H or an alkyl chain having from 1-24 carbon atoms, such as from 6-20 carbon atoms, or from 8-16 carbon atoms, said chain being saturated or unsaturated, linear or branched, substituted or unsubstituted.

In various embodiments, RCO— represents the coconut acid moiety. Non-limiting examples of acyl taurate salts include sodium cocoyl taurate and sodium methyl cocoyl taurate.

### Acyl Glycinates

Non-limiting examples of useful acyl glycinates include those of formula (X):

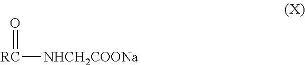

wherein R is an alkyl chain of 8 to 16 carbon atoms. Sodium is shown as the cation in the above formula (X), but the cation may be any alkali metal ion such as sodium or potassium, ammonium ions, or alkanolammonium ions such as monoethanolammonium or triethanolammonium ions.

Non-limiting examples of acyl glycinates include sodium cocoyl glycinate, sodium lauroyl glycinate, sodium myristoyl glycinate, potassium lauroyl glycinate, and potassium cocoyl glycinate, and in particular sodium cocoyl glycinate.

### Acyl Glutamates

Non-limiting examples of useful acyl glutamates include those of formula (XI):

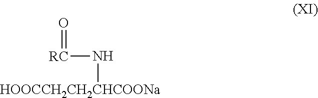

wherein R is an alkyl chain of 8 to 16 carbon atoms. Sodium is shown as the cation in the above formula (XI) but the cation may be any alkali metal ion such as sodium or potassium, ammonium ions, or alkanolammonium ions such as monoethanolammonium or triethanolammonium ions.

Non-limiting examples of acyl glutamates include dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, triethanolamine mono-cocoyl glutamate, triethanolamine lauroylglutamate, and disodium cocoyl glutamate.

In an embodiment, the non-sulfate anionic surfactants of the foaming cleansing compositions of the present disclosure may be selected from one or more acyl isethionates, their salts, or mixtures thereof.

In an embodiment, the one or more acyl isethionates, their salts, or mixtures thereof are selected from sodium cocoyl isethionate, sodium lauroyl methyl isethionate, sodium cocoyl methyl isethionate, sodium lauroyl isethionate, or mixtures thereof.

In an embodiment, the non-sulfate anionic surfactants of the foaming cleansing compositions of the present disclosure are selected from one or more acyl sarcosinates, their salts, or mixtures thereof.

In an embodiment, the one or more acyl sarcosinates, their salts, or mixtures thereof are selected from sodium cocoyl sarcosinate, sodium lauroyl sarcosinate, potassium lauroyl sarcosinate, potassium cocoyl sarcosinate, sodium myristoyl sarcosinate, sodium oleoyl sarcosinate, sodium palmitoyl sarcosinate, ammonium lauroyl sarcosinate, or mixtures thereof.

For example, two or more, such as three or more non-sulfate anionic surfactants may be selected from acyl isethionates, acyl sarcosinates, alkyl sulfonates, alkyl sulfosuccinates, alkyl sulfoacetates, alkoxylated monoacids, and acyl amino acids such as acyl taurates, acyl glycinates, acyl glutamates, salts thereof, or mixtures thereof.

### Sulfate Anionic Surfactants

Optionally, anionic surfactants may be chosen from sulfate anionic surfactants. Such sulfate anionic surfactants may be chosen from, for example, alkyl sulfates, alkyl ether sulfates, and/or salts thereof.

By way of example, alkyl sulfates may include C₈₋₁₈ alkyl sulfates, more C₁₂₋₁₈ alkyl sulfates, such as in the form of a salt with a solubilizing cation such as sodium, potassium, ammonium, or substituted ammonium. Examples include but are not limited to sodium lauryl sulfate (SLS) and sodium dodecyl sulfate (SDS).

As a further example, alkyl ether sulfates may include those having the formula:

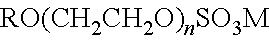

wherein R is an alkyl or alkenyl having from 8 to 18 (12 to 18) carbon atoms; n is a number having an average value of greater than at least 0.5, between 1 and 3, more between 2 and 3; and M is a solubilizing cation such as sodium, potassium, ammonium or substituted ammonium.

An example of a useful alkyl ether sulfate is sodium lauryl ether sulfate (SLES).

In some instances, useful alkyl sulfate salts and alkyl ether sulfate salts include those having the formulas (XII and XIII):

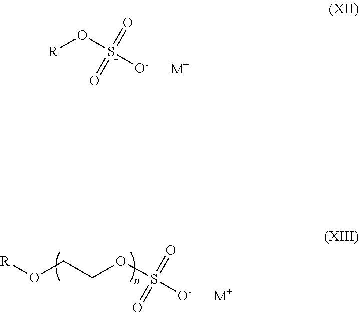

wherein R is an alkyl chain having from 6 to 24 carbon atoms, 8 to 18 carbon atoms, and more 12 to 18 carbon atoms; M is a solubilizing cation such as alkali metal ions such as sodium or potassium, ammonium ions, or alkanolammonium ions such as monoethanolammonium or triethanolammonium ions; and n is an integer from 0 to 3.

In some embodiments, foaming cleansing composition according to the disclosure does not require silicones and/or sulfate-based anionic surfactants. Thus, any one or more (or all) of these may optionally be excluded from the foaming cleansing compositions. In other words, the foaming cleansing compositions may be free or essentially free of silicones and/or sulfate-based anionic surfactants. Nonetheless, in some instances, one or more silicones and/or one or more sulfate-based surfactants may optionally be included in at least certain embodiments of the foaming cleansing compositions.

Thus, in various embodiments, one or more additional anionic surfactant may be present in a composition according to the disclosure from 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 120, 21, 22, 23, 24, 25, 26, 27, 28, 29, 30, 3, 32, 33, 34, 35, 36, 37, 38, 39, 40, 41, 42, 43, 44, 45, 46, 47, 48, 49 to about 50 percent, including increments and ranges therein and there between.

### Water

In the various embodiments, the foaming cleansing composition includes water.

In various embodiments, the foaming cleansing composition comprises from about 40% to about 90% water, and In some embodiments, from about 50% to about 85% water, and In some embodiments, from about 70% to about 80% water, including increments and all ranges and subranges therein and there between, by weight, based on the total weight of the foaming cleansing composition. One of ordinary skill in the art, however, will appreciate that other ranges are within the scope of the invention. Thus, water is present, by weight, based on the total weight of the foaming cleansing composition, from about 40, 45, 50, 55, 60, 65, 66, 67, 68, 69, 70, 71, 72, 73, 74, 75, 80, 85, to about 90 weight percent, including increments and all ranges and subranges therein and there between. In some embodiments, water is present at about 74%.

The water used in the foaming cleansing composition may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. The water phase may also comprise reconstituted thermal water, that is to say a water comprising trace elements such as zinc, copper, magnesium, etc., reconstituting the characteristics of a thermal water.

### Water-Soluble Solvents

In some embodiments, the foaming cleansing composition may include at least one water-soluble solvent. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90% in water under these conditions. Non-limiting examples of water-soluble solvents include, for example, glycerin, alcohols (for example, C1-C30, C1-C15, C1-C10, or C1-C4 alcohols), polyols, glycols, and combinations thereof.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and combinations thereof.

In some cases, a water-soluble solvent may comprise glycerin.

In the various embodiments total amount of the at least one water-soluble solvent, when present, may vary, is from about 0.5% to about 25%, or from about 0.5% to about 20%, or from about 1% to about 20%, or from about 1% to about 10%, or from about 2% to about 5% or any suitable combination, sub-combination, range, or sub-range thereof by weight, based on the weight of the foaming cleansing composition. One of ordinary skill in the art, however, will appreciate that other ranges are within the scope of the invention.

Thus, any one water-soluble solvent, when present, is present, by weight, based on the total weight of the foaming cleansing composition, from about 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 to about 25 weight percent, including increments and ranges therein and there between.

The pH of the foaming cleansing composition is not limited but is generally from 2 and 12, and in some embodiments, is one of from 3 and 11, and from 5 and 9, and from 6 and 8, and In some embodiments, is 7. In some particular embodiments, the pH of the foaming cleansing composition is in the range from about pH 4 to about pH 7.

The pH can be adjusted to the desired value by addition of a base (organic or inorganic) to the foaming cleansing composition, for example ammonia or a primary, secondary or tertiary (poly)amine, such as monoethanolamine, diethanolamine, triethanolamine, isopropanolamine or 1,3-propanediamine, or alternatively by addition of an inorganic or organic acid, advantageously a carboxylic acid, such as, for example, citric acid.

In some embodiments, the foaming cleansing composition excludes alcohols, for example, monoalcohols such as monohydric C₁-C₈ alcohols such as ethanol, propanol, butanol, isopropanol, isobutanol, and benzyl alcohol, and phenylethyl alcohol.

### Preservatives

In some embodiments, one or more preservatives and/or antimicrobials may be present in the foaming cleansing composition.

Any preservative commonly used in foaming cleansing compositions is an acceptable preservative for the foaming cleansing compositions herein, such as phenoxyethanol, members from the paraben family such as the methyl, ethyl, propyl, butyl or isobutyl parabens, 4-hydroxy benzoic acid, benzoic acid, sorbic acid, dehydroacetic acid, triclosan, benzyl alcohol, chlorophenesin, or salicylic acid, for example. At more concentrated amounts of suitable solvents for optional additives, in particular, suitable solvents for antimicrobials and preservatives, members from the paraben family may be used as a preservative.

In some embodiments, the one or more preservatives, when present, may be selected from phenoxyethanol, caprylyl glycol, Myrtrimonium bromide, hydroxyacetophenone, ethylhexyl glycerin, chlorphenesin, cetrimonium chloride, hexyl glycerin, octylglycerin, benzylglycerin, 3-heptoyl-2,2-propandiol, and 1,2-hexandiol; polyaminopropyl biguanide, also known as polyhexamethylene biguanide, or PHMB, and combinations thereof. In some embodiments, the foaming cleansing composition is free or essentially free of one or more of caprylyl glycol, phenoxyethanol, hexyl glycerin, ethylhexylglycerin, octylglycerin, benzylglycerin, 3-heptoyl-2,2-propandiol, and 1,2-hexandiol; polyaminopropyl biguanide, also known as polyhexamethylene biguanide, or PHMB.

In some embodiments, the preservative may comprise one or more of preservatives selected from the group consisting of organic acids, parabens, formaldehyde donors, phenol derivatives, quaternary ammoniums, alcohols, isothiazolinones, and combinations thereof. Preservatives having antibacterial activity are optionally present in the foaming cleansing compositions of the present invention. Examples of organic acid preservatives include, but are not limited to, sodium benzoate, potassium sorbate, benzoic acid and dehydroacetic acid, sorbic acid, and combinations thereof. A preferred organic acid preservative system includes a mixture of sodium benzoate and potassium sorbate. Examples of paraben preservatives include, but are not limited to, alkyl para-hydroxybenzoates, wherein the alkyl radical has from 1, 2, 3, 4, 5 or 6 carbon atoms and for example, from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben). Examples of formaldehyde donor preservatives include, but are not limited to, 1,3-Dimethylol-5,5-dimethylhydantoin (DMDM hydantoin), imidazolidinyl urea, gluteraldehyde, and combinations thereof. Examples of quaternary ammonium preservatives include, but are not limited to, benzalkonium chloride, methene ammonium chloride, benzethonium chloride, and combinations thereof. Examples of alcohol preservatives include, but are not limited to, ethanol, benzyl alcohol, dichlorobenzyl alcohol, phenoxyethanol, and combinations thereof. Examples of isothiazolone preservatives include, but are not limited to, methylchloroisothiazolinone, methylisothiazolinone, and combinations thereof.

In some embodiments, the preservative includes one or more preservatives, the one or combination present at a concentration, by weight of about 0.001% to about 5%, or alternatively about 0.05% to about 2.5% or alternatively about 0.1% to about 2.0%, or from about 0.02% to about 1% by weight, or from about 0.03% to about 0.5%, or any suitable combination, sub-combination, range, or sub-range thereof by weight, based upon weight of the foaming cleansing composition. One of ordinary skill in the art, however, will appreciate that other ranges are within the scope of the invention.

Thus, any one of or a combination of preservatives, when present, may be present, by weight, based on the total weight of the foaming cleansing composition, is from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 1.0, 1.1, 1.2, 1.3, 1.4, 1.5, 1.6, 1.7, 1.8, 1.9, 2, 3, 4, to about 5 weight percent, including increments and ranges therein and there between.

### Optional Additives

In some embodiments, the foaming cleansing composition includes one or more of other optional ingredients used in the cosmetics field selected from the group consisting of skin care actives, humectants, conditioning agents, thickeners, viscosity adjusters, cooling agents, fillers, antimicrobials, preservatives, pH adjusters, chelating agents, and combinations thereof, wherein the one or more of other optional ingredients does not adversely affect the properties of the foaming cleansing composition according to the invention.

In some particular embodiments, a composition according to the disclosure may include one or a combination of additives the group consisting of ceramide EOP, ceramide NP, ceramide AP, caprylyl glycol, phenoxyethanol, PEG-150 pentaerythrityl tetrastearate, carbomer, xanthan gum, propanediol, tetrasodium glutamate diacetate, sodium benzoate, phytosphingosine, glycerin, cholesterol, benzoic acid, sodium chloride, water, citric acid, triethyl citrate, sodium hyaluronate, niacinamide, and combinations thereof.

In some particular embodiments, a composition according to the disclosure may include one or more of rhamolipids and polylysine.

In some embodiments, a composition according to the disclosure may comprise one or a combination of additives selected from the group consisting of ceramide EOP (0%-1%), ceramide NP (0%-0.05%), ceramide AP (0%-0.05%), caprylyl glycol (0%-0.5%), phenoxyethanol (0%-2%), PEG-150 pentaerythrityl tetrastearate (0%-5%), carbomer (0%-0.1%), xanthan gum (0%-1%), propanediol (0%-20%), tetrasodium glutamate diacetate 0%-(0.5%), sodium benzoate (0%-1%), phytosphingosine (0%-0.05%), glycerin (0%-20%), cholesterol (0%-0.5%), benzoic acid (0%-0.5%), sodium chloride (0%-5%), citric acid (0%-1%), triethyl citrate (0%-0.5%), sodium hyaluronate (0%-1%), and niacinamide (0%-5.00%), and combinations thereof, the amounts representing an exemplary embodiment, by weight, based on the weight of the composition.

In some particular embodiments, a composition according to the disclosure may include one or more of rhamnolipids (0%-1.0%), polylysine (0%-0.1%), or a combination thereof, the amounts representing an exemplary embodiment, by weight, based on the weight of the composition.

In some embodiments, a composition according to the disclosure may comprise one or a combination of additives selected from the group consisting of ceramide EOP (0.250%), ceramide NP (0.0025%), ceramide AP (0.001%), caprylyl glycol (0.0015%), phenoxyethanol (0.50%), PEG-150 pentaerythrityl tetrastearate (0.50%), carbomer (0.0008%), xanthan gum (0.0008%), propanediol (3.00%), tetrasodium glutamate diacetate (0.07%), sodium benzoate (0.30%), phytosphingosine (0.00137500%), glycerin (5.00%), cholesterol (0.001%), benzoic acid (0.0006%), sodium chloride (0.9%), citric acid (0.73%), triethyl citrate (0.002%), sodium hyaluronate (0.001%), and niacinamide (2.00%), and combinations thereof, the amounts representing an exemplary embodiment, by weight, based on the weight of the composition.

In some particular embodiments, a composition according to the disclosure may include one or more of rhamnolipids (0.1-0.5%), polylysine (0.01%), or a combination thereof, the amounts representing an exemplary embodiment, by weight, based on the weight of the composition.

Although the aforementioned optional components are given as an example, it will be appreciated that other optional components compatible with cosmetic applications known in the art may be used.

In accordance with the various embodiments, the amount of one or more additional ingredients comprising actives or additives, alone or in combination, when present in the foaming cleansing composition according to the disclosure can be present in a range from about 0.0001% to about 20%, and in some embodiments, from about 0.005% to about 0.01%, and in some embodiments, from about 0.01% to about 0.1%, and in some embodiments, from about 0.15% to about 5%, and in some embodiments, from about 0.40% to about 4%, and in some embodiments, from about 0.5% to about 2.5%, and in some embodiments, from about 0.1% to about 0.5% and in some embodiments, from about 1% to about 2%, or any suitable combination, sub-combination, range, or sub-range thereof by weight, based on the total weight of the foaming cleansing composition. One of ordinary skill in the art, however, will appreciate that other ranges are within the scope of the invention.

Thus, any one or more additional ingredients comprising actives or additives may be present, each one or the combination present from about 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 percent, by weight, including increments and ranges therein and there between.

More generally, in some embodiments, a foaming cleansing composition includes at least one additive used in the cosmetics field which does not affect the properties of the foaming cleansing composition according to the invention, such as, fragrances, pH adjusters (citric acid, sodium chloride, lactic acid); neutralizing or pH-adjusting agents (e.g., triethylamine (TEA) and sodium hydroxide, Trisodium ethylenediamine disuccinate, and combinations thereof), other cosmetically acceptable additives, such as but not limited to, pearlescent agents, silica, and coloring materials; essential oils; fruit extracts, for example, Pyrus Malus (Apple) Fruit Extract, and Aloe Barbadensis Leaf Juice Powder; actives (for example, hydroxyacetophenone); vitamins (for example, vitamin A, beta carotene, tocopherol/vitamin E, panthenol, retinol, resveratrol, vitamin C, niacinamide, derivatives thereof, and combinations thereof); hyaluronic acid in the form of hydrolyzed hyaluronic acid or sodium hyaluronate; coloring materials/pigments; essential oils; antioxidants(phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria Baicalensis root extract), pine bark extract (Pinus Pinaster bark/bud extract), ellagic acid); vitamins and vitamin derivatives, such as calcium pantothenate, tocopherol and ascorbic acid; hydroxy acids; citric acid, sodium citrate, sodium chloride; neutralizing, chelating or pH-adjusting agents (for example, triethylamine (TEA), trisodium ethylenediamine disuccinate, EDTA, and sodium hydroxide); powders, fragrances, dyes, pigments; organic or mineral UV filters; conditioning agents such as C12-15 Alkyl lactate and C12-15 Alcohols; Thickeners, for example Ammonium polyacryloyldimethyl taurate, Propylene glycol and PEG-55 propylene glycol oleate; viscosity adjusters, for example, Sodium chloride and Hexylene Glycol; cooling agents such as menthol; fillers, for example Kaolin; or any combination thereof. Although the optional additives are given as examples, it will be appreciated that other optional components compatible with cosmetic applications known in the art may be used. Additives may also include humectants selected from glycerin, glycerol, butylene glycol, propylene glycol/propanediol, isoprene glycol, dipropylene glycol, hexylene glycol and polyethylene glycols, monoethylene glycol, diethylene glycol, triethylene glycol, diethylene glycol, hexylene glycol, glycol ethers such as monopropylene, dipropylene and tripropylene glycol alkyl(C1-C4)ethers, squalane, triacetin, sugars, such as glucose, xylitol, maltitol, sorbitol, sucrose pentaerythritol, inositol, pyrrolidone carboxylic acid, lactic acid, lithium chloride, acetamide MEA, sodium lactate, urea, dicyanamide, hyaluronic acid, aloe vera, honey, seaweed extract, or combinations thereof.

In some embodiments, there may be one or more actives selected from, for example, preservatives/anti-microbials (for example, chlorphenesin, salicylic acid, phenoxyethanol, potassium sorbate, and caprylyl glycol); actives (for example, hydroxyacetophenone); vitamins (for example, vitamin A, beta carotene, tocopherol/vitamin E, panthenol, retinol, resveratrol, vitamin C, niacinamide, derivatives thereof, and combinations thereof); hyaluronic acid in the form of hydrolyzed hyaluronic acid or sodium hyaluronate; coloring materials/pigments; essential oils; antioxidants(phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria Baicalensis root extract), pine bark extract (Pinus Pinaster bark/bud extract), ellagic acid); vitamins and vitamin derivatives, such as calcium pantothenate, tocopherol and ascorbic acid; hydroxy acids; citric acid, sodium citrate, sodium chloride; neutralizing, chelating or pH-adjusting agents (for example, triethylamine (TEA), trisodium ethylenediamine disuccinate, EDTA, and sodium hydroxide); powders, fragrances, dyes, pigments; organic or mineral UV filters; and combinations thereof.

In the various embodiments, the amount of one or more additives, alone or in combination, present in the foaming cleansing composition can be present in a range from about 0.0001% to about 20%, and in some embodiments, from about 0.005% to about 0.01%, and in some embodiments, from about 0.01% to about 0.1%, and in some embodiments, from about 0.15% to about 5%, and in some embodiments, from about 0.40% to about 4%, and in some embodiments, from about 0.5% to about 2.5%, and in some embodiments, from about 0.1% to about 0.5% and in some embodiments, from about 1% to about 2%, or any suitable combination, sub-combination, range, or sub-range thereof by weight, based on the total weight of the foaming cleansing composition. One of ordinary skill in the art, however, will appreciate that other ranges are within the scope of the invention.

Thus, any one or a combination of additives may be present, by weight, based on the total weight of the foaming cleansing composition, each one or the combination present from about 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 weight percent, including increments and ranges therein and there between.

In some embodiments, the foaming cleansing composition may include one or more oils. In some embodiments, the foaming cleansing composition is essentially free, free of or devoid of oils.

In some examples, the oils may be chosen from hydrocarbon-based oils. For example, the hydrocarbon-based oil may be a saturated hydrocarbon, an unsaturated hydrocarbon, lipids, triglycerides, a natural oil, and/or a synthetic oil. In some embodiments, the foaming cleansing composition may include a synthetic oil selected from the group consisting of hydrogenated polyisobutene and hydrogenated polydecene. A hydrocarbon-based oil may be a non-volatile hydrocarbon-based, such as:
(i) hydrocarbon-based oils of plant origin, such as glyceride triesters, which are generally triesters of fatty acids and of glycerol, the fatty acids of which can have varied chain lengths from C4 to C24, it being possible for these chains to be saturated or unsaturated and linear or branched; these oils are in particular wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, musk rose oil, and essential oils, such as Helianthus Annuus Seed Oil, Lavandula Angustifolia (lavender) Oil, Mentha Piperita Oil, Rosmarinus Officinalis (rosemary) Leaf Oil Pelargonium Graveolens flower oil, Citrus Aurantium Dulcis (orange) peel oil, Menthe Viridis (spearmint) leaf oil, Citrus Aurantifolia (lime) oil, Melaleuca Alternifolia (tea tree) leaf oil, Citrus Grandis (grapefruit) peel oil, Citrus Medica Limonum (lemon) peel oil, rose flower oil, eucalyptus globulus leaf oil, and combinations thereof.
(ii) synthetic ethers containing from 10 to 40 carbon atoms;
(iii) linear or branched hydrocarbons of mineral or synthetic origin, such as petroleum jelly, polydecenes, hydrogenated polyisobutene such as Parleam, and 40 squalane;
(iv) synthetic esters, for instance oils of formula RCOOR′ in which R represents a linear or branched fatty acid residue containing from 1 to 40 carbon atoms and R′ represents a hydrocarbon-based chain that is especially branched, containing from 1 to 40 carbon atoms on condition that R+R′ is ÿ 10, for instance Purcellin oil (cetearyl octanoate), isopropyl myristate, isopropyl palmitate, C12-C15 alkyl benzoate, such as the product sold under the trade name Finsolv TN™ or Witconol TN™ by Witco or Tegosoft TN™ by Evonik Goldschmidt, 2-ethylphenyl benzoate, such as the commercial product sold under the name X-Tend 226 by ISP, isopropyl lanolate, hexyl laurate, diisopropyl adipate, isononyl isononanoate, oleyl erucate, 2-ethylhexyl palmitate, isostearyl isostearate, diisopropyl sebacate, such as the product sold under the name of “Dub Dis” by Stearinerie Dubois, octanoates, decanoates or ricinoleates of alcohols or polyalcohols, such as propylene glycol dioctanoate; hydroxylated esters, such as isostearyl lactate or diisostearyl malate; and pentaerythritol esters; citrates or tartrates, such as di(linear C12-C13 alkyl) tartrates, such as those sold under the name Cosmacol ETI™ by Enichem Augusta Industriale, and also di(linear C14-C15 alkyl) tartrates, such as those sold under the name Cosmacol ETL™ by the same company; or acetates;
(v) fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;
(vi) higher fatty acids, such as oleic acid, linoleic acid or linolenic acid;
(vii) carbonates, such as dicaprylyl carbonate, such as the product sold under the name Cetiol CC™ by Cognis;
(viii) fatty amides, such as isopropyl N-lauroyl sarcosinate, such as the product sold under the trade name Eldew SL 205™ from Ajinomoto; and
(ix) essential oils selected from the group consisting of sunflower oil, sesame oil, peppermint oil, macadamia nut oil, tea tree oil, evening primrose oil, sage oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang.

In some embodiments, the foaming cleansing composition may include at least one branched or linear liquid alkane with carbon chain length of C11 to C20. In various embodiments, liquid alkanes may be selected from those with a carbon chain length of from C11 to C20. The liquid alkanes may be selected from those with a carbon chain length of from C11 to C20, or from C15 to C19, or one of C11, C12, C13, C14, C15, C16, C17, C18 to C19. In some particular embodiments, suitable liquid alkanes that may be used according to the disclosure include hydrocarbon-based oils containing from 8 to 16 carbon atoms, and especially branched C8-C16 alkanes such as C8-C16 isoalkanes. In some exemplary embodiments, such liquid alkanes may be chosen from isoparaffins, for instance isododecane (also known as 2,2,4,4,6-pentamethylheptane), isodecane, and isohexadecane.

The amount of each of the at least one branched or linear liquid alkane may be present in the foaming cleansing composition in a range of from about 1% to about 35% by weight, or from about 15% to about 30% by weight, or from about 10% to about 20% or any suitable combination, sub-combination, range, or sub-range thereof by weight, based on the weight of the foaming cleansing composition. One of ordinary skill in the art, however, will appreciate that other ranges are within the scope of the invention.

Thus, each of the at least one branched or linear liquid alkane in the foaming cleansing composition may be present by weight, based on the total weight of the foaming cleansing composition, from about 1, 2, 3, 4, 5, 6, 7, 8, 9, 10, 11, 12, 13, 14, 15, 16, 17, 128, 19, 20, 21, 22, 23, 24, 25, 26, 27, 28, 29, 30, 31, 32, 33, 34, to about 35 percent, including increments and ranges therein and there between.

Although the optional additives are given as examples, it will be appreciated that other optional components compatible with cosmetic applications known in the art may be used that are suitable. It will be appreciated by a skilled artisan that any optional additives are present only to the extent and in amounts that do not materially adversely affect the basic and novel characteristic(s) of the claimed disclosure. Thus, In some embodiments, that include optional additives, such optional additives will not materially adversely affect the foaming cleansing composition.

More generally, it will be appreciated by a skilled artisan that any solvents, humectants, preservatives or other additives are present only to the extent and in amounts that do not materially adversely affect the basic and novel characteristic(s) of the claimed invention.

### EXAMPLE

### Example 1: Compositions

Various representative embodiments of the inventive and comparative compositions are exemplified herein.

TABLE 2
Inventive and Comparative Compositions

| INCI | INV 1 | COMP | INV 2 | INV 3 | INV 4 |
|---|---|---|---|---|---|
| COCAMIDOPROPYL | 5.00 | 5.00 | 5.00 | 5.00 | 5.00 |
| HYDROXYSULTAINE | | | | | |
| COCO-BETAINE | 3.00 | 3.00 | 3.00 | 3.00 | 3.00 |
| SODIUM METHYL COCOYL | 0.90 | 0.00 | 0.90 | 0.90 | 0.90 |
| TAURATE | | | | | |
| SODIUM COCOYL | 4.35 | 4.35 | 2.55 | 1.80 | |
| SARCOSINATE | | | | | |
| SODIUM LAUROYL | | | 1.86 | 2.635 | 4.495 |
| SARCOSINATE | | | | | |
| SODIUM LAUROYL | 0.025 | 0.025 | 0.025 | 0.025 | 0.025 |
| LACTYLATE | | | | | |
| PEG-150 PENTAERYTHRITYL | 0.50 | 0.50 | 0.50 | 0.50 | 0.50 |
| TETRASTEARATE | | | | | |
| sodium lauroyl lactylate, ceramide | ~13 | ~13 | ~13 | ~13 | ~13 |
| EOP, ceramide NP, ceramide AP, | | | | | |
| caprylyl glycol, phenoxyethanol, | | | | | |
| carbomer, xanthan gum, | | | | | |
| propanediol, tetrasodium glutamate | | | | | |
| diacetate, sodium benzoate, | | | | | |
| phytosphingosine, glycerin, | | | | | |
| cholesterol, benzoic acid, sodium | | | | | |
| chloride, citric acid, triethyl citrate, | | | | | |
| sodium hyaluronate, niacinamide | | | | | |
| WATER | ~74/QS | ~75/QS | ~74/QS | ~74/QS | ~74/QS |

TABLE 2
Commercial Benchmark Composition

| INCI US | Benchmark |
|---|---|
| COCAMIDOPROPYL HYDROXYSULTAINE | 6.48 |
| SODIUM METHYL COCOYL TAURATE | 1.00 |
| SODIUM LAUROYL SARCOSINATE | 4.47 |
| SODIUM LAUROYL LACTYLATE | 0.025 |
| PEG-150 PENTAERYTHRITYL TETRASTEARATE | 2.75 |
| PEG-6 CAPRYLIC/CAPRIC GLYCERIDES | 1.10 |
| WATER | ~75 |
| ceramide EOP, ceramide NP, ceramide AP, carbomer, | ~9.4 |
| xanthan gum, disodium EDTA, phytosphingosine, | |
| glycerin, propylene glycol, cholesterol, tetrasodium | |
| EDTA, sodium chloride, citric acid, hydrolyzed | |
| hyaluronic acid, niacinamide, propylparaben | |
| methylparaben | |

### Example 2: Performance of Compositions

TABLE 3
Performance of Inventive and Comparative Compositions

| | Inventive 1 | Comparative | Inventive 2 | Inventive 3 | Inventive 4 |
|---|---|---|---|---|---|
| 55 C. 1 week | Clear | Clear | Clear | CLEAR | Slight |
| | | | | | Sediment |
| 55 C. 2 weeks | Clear | Slight | Clear | Slight | Slight |
| | | Sediment | | Sediment | Sediment |
| 3 week | Clear | Sediment | Clear | Sediment | Slight |
| | | | | | Sediment |
| VI | 32.5 | 21.7 | 36.1 | 28.7 | 33.6 |
| pH | 5.0 | 5.0 | 5.0 | 5.0 | 5.0 |
| 1 month 45 C. | 27.2 | 16.8 | 18 | 17.4 | 17.8 |
| 2 month 45 C. | 17.7 | 8.02 | 14 | 13.4 | 16.4 |
| Overall stability | Excellent | Poor/Fail | Good | Good | Good |

Viscosity is in units of UD

The foregoing demonstrates that the inventive compositions demonstrate good to excellent overall stability, even after exposure to high heat for up to two months. Note, Comparative comparison lacks the taurate anionic surfactant, its performance evidencing the importance of the claimed combination of anionic surfactants, in contrast to the strong stability and viscosity performance of Inventive 1 which includes only sodium cocoyl sarcosinate as a second anionic surfactant together with the taurate anionic surfactant, inventives 2 and 3 which include a blend of sodium cocoyl sarcosinate and sodium lauroyl sarcosinate, and inventive 4 which includes sodium lauroyl sarcosinate in the absence of sodium cocoyl sarcosinate

### Example 3: Antimicrobial Performance

TABLE 4
Antimicrobial performance

| | Microbiome | | | | | | | | | |
|---|---|---|---|---|---|---|---|---|---|---|
| | Killing | | | | | | | | | |
| | C Acnes | | S Aureus | | | | S Epidermidis | | | |
| | 2 h | 6 h | 2 h | | 6 h | | 2 h | | 6 h | |
| Benchmark | −5.2 Log | −5.2 Log | −0.3 | Log | −1.7 | Log | 0 | | 0 | |
| Inventive 1 | −5.2 Log | −5.2 Log | −2 | log | −3.3 | log | −0.3 | log | −2 | log |
| Inventive 2 | −5.2 Log | −5.2 Log | −1.8 | log | −3.3 | log | −0.3 | log | −2.5 | log |

The foregoing demonstrates that in particular inventives 1 and 2 demonstrate excellent and maximal against C. acnes from 2 h of contact time (5.1 Log reduction at 2 h); excellent against S. aureus at 24 h of contact time (4.60 Log reduction at 24 h); excellent against S. epidermidis at 24 h of contact time (3.37 Log reduction at 24 h), with particular improvement over the benchmark with respect to activity against S. epidermidis.

### Example 4: Effect of Certain Additional Ingredients

TABLE 5
Addition of Ceramides, Rhamnolipids, Polylysine in Inventive #1

| | Inventive 1 A | Inventive 1B | Inventive 1C | Inventive 1D |
|---|---|---|---|---|
| Ceramide | 0.0375 | 0 | 0.015 | 0.015 |
| Rhamnolipid | 0.5 | 0.5 | 0.1 | 0.5 |
| Polylysine | 0.01 | 0.01 | 0.01 | 0.01 |
| Viscosity | 25 | 18.1 | 20 | 14.4 |
| pH | 5.06 | 5.04 | 5.04 | 5.04 |
| 55 C. 1 wk | Sediment | Barely sediment | Barely sediment | Clear |
| 55 C. 2 wk | — | — | — | — |
| 55 C. 3 wk | Sediment | Barely Sediment | Barely Sediment | Clear |

The foregoing demonstrates that inclusion of ceramide, rhamnolipid and polylysine can be particularly beneficial to the stability of the composition

The results demonstrate that the inventive composition performs at least as well as the commercial benchmark and has more desirable foaming and textural/viscosity properties.

While the disclosure has been described with reference to an embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

The articles “a” and “an,” as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The transitional terms “comprising,” “consisting essentially of” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed disclosure. All materials and methods described herein that embody the present disclosure can, in alternate embodiments, be more specifically defined by any of the transitional terms “comprising,” “consisting essentially of,” and “consisting of.”

“Free” and “devoid” and “exclude” or “excluded” each indicates that no reliably measurable excluded material, for example, an excluded thickener or excluded sulfate surfactant or other excluded material as described herein, is present in the composition. The term “essentially free” means that, while it is preferred that no excluded material is present in the composition, it is possible to have very small amounts of the excluded material in the composition of the invention, provided that these amounts do not materially affect the advantageous properties of the composition. In particular, “essentially free” means that excluded material can be present in the composition at an amount of less than 5%, or less than 4%, or less than 3%, or less than 2%, or less than 1%, or less than 0.1% by weight, or at 0%, based on the total weight of the composition.

In accordance with the various embodiments, it is contemplated that some or all of following ingredients may be excluded from the composition according to the invention: alcohols, for example but not limited to, monohydric alcohols; powders comprising Nylon-12, polymethyl methacrylate, polyethylene beads, and powders which are considered microplastics; lactic acid, glycolic acid, tartaric acid, mandelic acid, citric acid, salicylic acid and derivatives thereof (including 5-n-octanoylsalicylic acid, salicylate, sodium salicylate, and willow extract), salicylic acid, capryloyl salicylic acid, beta hydroxybutanoic acid, propionic acid, beta-hydroxy beata-methylbutyric acid, carnitine tropic acid, and trethocanic acid; alkyl polyglucosides selected from the group consisting of decyl glucoside, lauryl glucoside, octyl glucoside, coco glucoside, caprylyl/capryl glucoside, and sodium lauryl glucose carboxylate, and combinations thereof; surfactants with a C10-C20 fatty alcohol or acid hydrophobe condensed with from about 2 to about 100 moles of ethylene oxide or propylene oxide per mole of hydrophobe; C2-C10 alkyl phenols condensed with from 2 to 20 moles of alkylene oxide; mono- and di-fatty acid esters of ethylene glycol; fatty acid monoglyceride; sorbitan; mono- and di-C8-C20 fatty acids; polyoxyethylene sorbitan; alkyl polyglycosides and saccharide fatty amides (e.g. methyl gluconamides); alkyl ether sulfate and sulfonates; alkyl sulfates and sulfonates; alkylbenzene sulfonates; alkyl and dialkyl sulfosuccinates; C8-C20 acyl isethionates; C8-C20 alkyl ether phosphates; alkylethercarboxylate, PEG-100 Stearate; PEG-20 Stearate and other esters of Poly(Ethylene Glycol); polyethylene glycol (PEG) surfactants, polypropylene glycol (PPG) surfactants, sulfate surfactants; taurates, for example, taurate surfactants; Sucrose Stearate and other emulsifiers based on sugar esters; Glyceryl Stearate and other glycerol esters; Disodium Ethylene Dicocamide PEG-15 Disulfate; Sodium Steroyl Glutamate and other fatty amides; Steareth-100 and other fatty ethers, cationic surfactants, and other cationic compounds; silicones, such as silicone polymers, silicone elastomers, and silicone oils, for example selected from dimethicone, cyclopentasiloxane, cyclohexasiloxane, and combinations thereof; phthalates, parabens, sulfates, polyquaternium, microplastics, synthetic dyes, gelling agents, and combinations thereof.

“Excluded polymeric thickeners” means and refers to thickeners that have not been added as a component in the composition or that may be excluded in some embodiments. Thus, in some embodiments, the composition is essentially free, free or devoid of one or more of PEG/PEO polymeric thickeners, nature based thickeners, and synthetic thickeners. In some embodiments, the composition is essentially free of or excludes PEG-type polymeric thickeners selected from PEG-150 pentaerythrityl tetrastearate, PEG-120 thickeners, PEG-150 thickeners, PEG-80 glyceryl tallow ester, PEG-8 PPG (polypropylene glycol)-3 diisostearate, PEG-200 hydrogenated glyceryl palmitate, PEG-n (n=6, 8, 12) beeswax, PEG-4 isostearate, PEG-n (n=3, 4, 8, 150) distearate PEG-n (n=28, 200) glyceryl tallow ester, PEG-7 hydrogenated castor oil, PEG-40 jojoba oil, PEG-120 methyl glucose dioleate, PEG-150 pentaerythritol stearate, PEG-55 propylene glycol oleate, PEG-160 sorbitan triisostearate, SMDI copolymer (polyethylene glycol-150/decyl/methacrylate copolymer), PEG-150/stearyl/SMDI copolymer, PEG-90, and combinations thereof. In some embodiments, the composition may exclude one or more polymers such as cationic polymers selected from polylysine, chitosan, and guar. In some embodiments, the composition may exclude one or more polymers selected from polysaccharides that include alginates/algin, chitosan, cyclodextrin, cationic gelatin, cationic dextran, celluloses, cationic cellulose, chitin, starches, galactomannans such as guar gums and its derivatives, including hydroxypropyl guar, cationic guar derivatives, gums of microbial origin, including xanthan gum, scleroglucan gum, mucopolysaccharides, chondroitin sulfates, and mixtures thereof, and gums derived from plant exudates, including locust bean gums, gum arabic, ghatti gum, karaya gum, gum tragacanth, carrageenan gum, agar gum and carob gum, cellulose-based polymers, including methylcellulose, hydroxyalkylcellulose, ethylhydroxyethylcelluloses, hydroxyethylcellulose, hydroxypropylcellulose, carboxymethylcellulose, cationic cellulose ether derivatives, quaternized cellulose derivatives, nitrocellulose, hemicellulose and hemicellulose derivatives, mannans, xylans, lignins, arabans, galacturonans, alginate-based compounds, glucuronoxylans, arabinoxylans, xyloglucans, glucomannans, fructosans such as inulin, pectic acids and pectins, arabinogalactans, agars, glycosaminoglycans, gelatin, gellan, crosslinked homopolymers of acrylic acid, cationic peptides and their derivatives (e.g., polylysine, polyomithine), peptide/protein polymers (e.g., histone, collagen); cationic thiolated biopolymers (nature-based thiomers or nature-based dendrimers, e.g., chitosan-cysteine, chitosan-thiobutylamidine, chitosan-thioglycolic acid); algin, xanthan gum (and) sclerotium gum (and) lecithin (and) pullulan (Siligel™), xanthan gum, carbomer 980, hydroxypropyl starch phosphate, ammonium polyacryloyldimethyl taurate, hydroxyethyl cellulose, acrylate copolymer, sclerotium gum; or combinations thereof. In some embodiments, the composition may also exclude one or more PEG type compound selected from poloxamer 184 (polyoxyethylene polyoxypropylene glycol), PEG-20 glyceryl triisostearate, PEG-7 glyceryl cocoate, PEG-20 methylglucoside sesquistearate, PG-5 dioleate, PG-4 diisostearate, PG-10 isostearate, PEG-60 hydrogenated castor oil, PEG-8 stearate, PEG-8 isostearate, PEG-60 hydrogenated castor oil, PEG-150 Laurate, PEG-150 Distearate, PEG-150 pentaerythrityl tetrastearate, PEG-78 Glyceryl Cocoate, PEG-30 Glyceryl Cocoate, PEG-type polymeric thickeners selected from PEG-150 pentaerythrityl tetrastearate, PEG-120 thickeners, PEG-150 thickeners, PEG-80 glyceryl tallow ester, PEG-8 PPG (polypropylene glycol)-3 diisostearate, PEG-200 hydrogenated glyceryl palmitate, PEG-n (n=6, 8, 12) beeswax, PEG-4 isostearate, PEG-n (n=3, 4, 8, 150) distearate PEG-n (n=28, 200) glyceryl tallow ester, PEG-7 hydrogenated castor oil, PEG-40 jojoba oil, PEG-120 methyl glucose dioleate, PEG-150 pentaerythritol stearate, PEG-55 propylene glycol oleate, PEG-160 sorbitan triisostearate, SMDI copolymer (polyethylene glycol-150/decyl/methacrylate copolymer), PEG-150/stearyl/SMDI copolymer, PEG-90, -20, Ceteth-10, Ceteth-20, Isoceteth-20, Laureth-4, Laureth-23, Oleth-10, Oleth-20, Steareth-10, Steareth-20, Steareth-100, and Steareth-21, PEG-20 glyceryl laurate, PEG-40 hydrogenated castor oil, or combinations thereof.

“Sulfate surfactant-free” means that excluded surfactants have not been added as a component. In some particular embodiments, the composition is devoid of sulfate based surfactant. In some embodiments, the composition is devoid of ethylene glycol (PEG) based surfactants. Those of skill in the art will appreciate that a surfactant may be present in a composition via its presence in one or more of the formulation components; thus, In some embodiments, the composition may be “essentially-free” wherein the excluded surfactant is present at a concentration that does not exceed 5% by weight, and in some instances is present not more than 3% by weight, and in some instances is present not more than 1% by weight, based on the weight of the cosmetic composition. In some particular embodiments “sulfate surfactant-free” means that the cosmetic composition is free or devoid specifically of the excluded sulfate surfactant.

“Petrochemical-free” means that an excluded petrochemical has not been added as a component. Some specific but non-limiting examples of petrochemicals that are lacking from the cosmetic composition include benzalkonium chloride, isododecane, isohexadecane and the like. In some embodiments, the composition is not free from petrochemicals, but is formulated to have an amount of petrochemicals that is at or below the amounts found in similar compositions that are commercially available. The composition may be petrochemical-free.

“Silicone-free” means that an excluded silicones has not been added as a component. In some embodiments, a composition is devoid of silicones. Some specific but non-limiting examples of silicones that are lacking from the cosmetic composition includes, but is not limited to, silicone polymers, for example selected from dimethicone, cyclopentasiloxane, and other silicone oils, and silicone elastomers. The composition may be silicone-free.

Other than in the operating examples, or where otherwise indicated, all numbers expressing quantities of ingredients and/or reaction conditions may be understood as being modified in all instances by the term “about,” meaning within 10% of the indicated number (e.g., “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

What is claimed is:

1) A foaming cleansing composition, comprising:
a) a blend of surfactants comprising:
i) a combination of at least two amphoteric surfactants, including cocoamidopropyl hydroxysultaine and coco betaine, present in a total amount of about 8% by weight, based on the weight of the composition; and
ii) a combination of at least two anionic surfactants, comprising sodium methyl cocoyl taurate and at least one of sodium cocoyl sarcosinate or sodium lauroyl sarcosinate, each present in an amount of at least 1.8% by weight or 1.9% by weight, based on the weight of the composition, respectively; and
b) water,
wherein the composition demonstrates long-term stability at elevated temperatures, remaining visibly clear with stable viscosity, antimicrobial resistance, and resistance to cloudiness and sedimentation at ambient and high temperatures.

2) The foaming cleansing composition according to claim 1, wherein cocoamidopropyl hydroxysultaine is present in a range from about 1% to about 20% and coco betaine is present in a range from about 1% to about 15%, all amounts by weight, based on the weight of the composition.

3) The foaming cleansing composition according to claim 1, wherein cocoamidopropyl hydroxysultaine is present in a range from about 3% to about 7%, and coco betaine is present in a range from about 1% to about 5%, all amounts by weight, based on the weight of the composition.

4) The foaming cleansing composition according to claim 1, wherein water is present in a range from about 40% to about 90%, by weight, based on the weight of the foaming cleansing composition.

5) The foaming cleansing composition according to claim 1, wherein sodium methyl cocoyl taurate is present from about 0.1% to about 1.5%, by weight, based on the weight of the composition.

6) The foaming cleansing composition according to claim 1, wherein sodium cocoyl sarcosinate is present in an amount from about 1.8% to about 10%, by weight, based on the weight of the composition.

7) The foaming cleansing composition according to claim 1, comprising sodium cocoyl sarcosinate wherein the ratio of sodium cocoyl sarcosinate to sodium methyl cocoyl taurate is in a range from about 1:1 to about >1:1 by weight of each ingredient, or comprising or sodium lauroyl sarcosinate wherein the ratio of sodium lauroyl sarcosinate to sodium methyl cocoyl taurate is in a range from about 1:1 to about >1:1 by weight of each ingredient.

8) The foaming cleansing composition according to claim 1, wherein sodium lauroyl sarcosinate is present from about 1.9% to about 6%, by weight, based on the weight of the composition.

9) The foaming cleansing composition according to claim 1, comprising at least one additional anionic surfactant comprising sodium isethionate is present from about 0.001% to about 2%, by weight, based on the weight of the composition.

10) The foaming cleansing composition according to claim 1, comprising at least one additional anionic surfactant comprising sodium lauroyl lactylate is present from about 0.015% to about 0.035%, by weight, based on the weight of the composition.

11) The foaming cleansing composition according to claim 1, which demonstrates foaming, viscosity, and solubility stability at ambient and elevated temperatures, and antimicrobial activity against C. acnes, S. aureus, and S. epidermidis.

12) The foaming cleansing composition according to claim 1, comprising at least one or a combination of polylysine, ceramide, and glycolipids.

13) The foaming cleansing composition according to claim 1, comprising a glycolipid selected from the group consisting of rhamnolipids, trehalolipids, sophorolipids, mannosylerythritol lipids, and combinations thereof.

14) The foaming cleansing composition according to claim 1, comprising a glycolipid comprising at least one rhamnolipid.

15) The foaming cleansing composition according to claim 1, comprising one or more ingredients selected from the group consisting of ceramide EOP, ceramide NP, ceramide AP, caprylyl glycol, phenoxyethanol, PEG-150 pentaerythrityl tetrastearate, carbomer, xanthan gum, propanediol, tetrasodium glutamate diacetate, sodium benzoate, phytosphingosine, glycerin, cholesterol, benzoic acid, sodium chloride, water, citric acid, triethyl citrate, sodium hyaluronate, niacinamide.

16) The foaming cleansing composition according to claim 1, which comprises PEG-150 pentaerythrityl tetrastearate in an amount that is less than 1%, by weight, based on the weight of the composition.

17) The foaming cleansing composition according to claim 1, which excludes PEG/PEO polymeric thickeners, parabens, and combinations thereof.

18) The foaming cleansing composition according to claim 1, which excludes PEG-150 pentaerythrityl tetrastearate, PEG-6 caprylic/capric glycerides, or a combination thereof.

19) A foaming cleansing composition, comprising:
a) a blend of surfactants comprising:
i) cocoamidopropyl hydroxysultaine in a range from about 3% to about 7%; and
ii) a combination of at least two anionic surfactants, including sodium methyl cocoyl taurate present from about 0.1% to about 1.5%, and sodium cocoyl sarcosinate present in an amount from about 1.8% to about 10%; and
b) water,
all amounts by weight, based on the weight of the composition,
wherein the composition demonstrates long-term stability at elevated temperatures, remaining visibly clear with stable viscosity, antimicrobial resistance, and resistance to cloudiness and sedimentation at ambient and high temperatures.

20) A foaming cleansing composition, comprising:
a) a blend of surfactants comprising:
i) a combination of at least two amphoteric surfactants, including cocoamidopropyl hydroxysultaine and coco betaine, present in a total amount of about 8% by weight; and
ii) a combination of at least two anionic surfactants, comprising sodium methyl cocoyl taurate and at least one of sodium cocoyl sarcosinate or sodium lauroyl sarcosinate, each present in an amount of at least 1.8% by weight or 1.9% by weight, respectively; and
b) water present in a range from about 40% to about 90%, by weight, based on the weight of the foaming cleansing composition;
c) one or more ingredients selected from the group consisting ceramide EOP, ceramide NP, ceramide AP, caprylyl glycol, phenoxyethanol, PEG-150 pentaerythrityl tetrastearate, carbomer, xanthan gum, propanediol, tetrasodium glutamate diacetate, sodium benzoate, phytosphingosine, glycerin, cholesterol, benzoic acid, sodium chloride, water, citric acid, triethyl citrate, sodium hyaluronate, niacinamide, and combinations thereof;
wherein the composition demonstrates foaming, viscosity, and solubility stability at ambient and elevated temperatures, and antimicrobial activity against C. acnes, S. aureus, and S. epidermidis, and wherein, if present, PEG-150 pentaerythrityl tetrastearate is present in an amount that is less than 1%,
all amounts by weight, based on the weight of the composition.
